(19) United States
(12) Patent Application Publication
(10) Pub. No.: US 2014/0121549 A1
(43) Pub. Date: May 1, 2014

(54) METHOD AND APPARATUS FOR DETERMINING THE MYOCARDIAL INOTROPIC STATE

(71) Applicant: KATHOLIEKE UNIVERSITEIT LEUVEN, KU LEUVEN R&D, (US)

(72) Inventors: Piet CLAUS, Jodoigne (BE); Jan D'hooge, Mechelen (BE); Ruta Jasaityte, Vilnius (LT); Frank Rademakers, Winksele (BE)

(73) Assignee: KATHOLIEKE UNIVERSITEIT LEUVEN, KU LEUVEN R&D, Leuven (BE)

(21) Appl. No.: 13/762,810

(22) Filed: Feb. 8, 2013

Related U.S. Application Data
(60) Provisional application No. 61/733,751, filed on Dec. 5, 2012.

(30) Foreign Application Priority Data
Oct. 31, 2012 (GB) 1219572.3

(51) Int. Cl. A61B 5/02; A61B 8/13; A61B 8/08; A61B 5/0452; A61B 5/04

(52) U.S. Cl. CPC A61B 5/02028; A61B 5/0452; A61B 5/04012; A61B 8/485; A61B 8/0883; A61B 8/13; A61B 8/5223; A61B 8/488
USPC 600/509; 600/508

(57) ABSTRACT

A method for determining myocardial inotropic state is described. The method involves receiving a value of stretch for different myocardial segments during passive filling, e.g. atrial contraction, and receiving associated values representative of total systolic strain. The method also involves using a relationship between the stretch of different myocardial segments during atrial contraction and the associated values representative for total systolic strain as an index of the myocardial inotropic state. A corresponding system and computer program product also is described.

### FIELD OF THE INVENTION

The present invention relates to a method and apparatus for determining myocardial inotropic state. More particularly, the present invention relates to methods and systems for determining myocardial inotropic state, e.g. LV inotropy, based on a non-invasive index. Embodiments relate to a method and apparatus for determining said myocardial inotropic state using a segmental stretch-strain relationship, e.g. use of the relationship between the differing stretch of myocardial segments across the cardiac chamber, e.g. left ventricle, during passive filling, e.g. atrial contraction, and associated values representative for, e.g. the related variance in, total systolic strain, as an index of the myocardial inotropic state.

### BACKGROUND OF THE INVENTION

Inotropy describes the intrinsic ability of myocardium to generate force independent of loading conditions. Clinically applicable measurements of inotropy could be very useful in various clinical settings like chronic heart failure and valvular heart disease. However, this remains a difficult task as non-invasive estimates of the left ventricular (LV) inotropic state are limited by their load dependency.

Currently, only invasive measurements, such as the end-systolic pressure volume relationship (ESPVR) and preload recruitable stroke work (PRSW), give a good estimate of myocardial inotropy. They both use the Frank-Starling mechanism which is known as a phenomenon where at a given inotropic state active force developed by the ventricle increases with increasing preload. As this systolic LV response to preload is also modulated by the inotropic state, varying preload and measuring the LV response to this intervention can be used to assess LV inotropy. Unfortunately, this approach towards an estimation of the cardiac inotropic state cannot be easily applied in the daily routine as it requires simultaneous invasive recordings of LV pressures and volumes under changing preload conditions. Moreover, varying preload and invasively measuring the LV pressure and volume response cannot easily be applied in heart failure patients that do not tolerate volume challenges. In addition, the basis of the FS mechanism is on a cellular level.

A similar relationship between diastolic preload and systolic LV response may be present on a regional level as well. It is known that regional myocardial stretch during atrial contraction and systolic LV strain are inhomogeneous and related to each other as disclosed by Zwanenburg et al in “Regional timing of myocardial shortening is related to prestretch from atrial contraction: assessment by high temporal resolution MRI tagging in humans” disclosed in the Am. J. Physiol Heart CircPhysiol, 288:H787-94 (2005). MRI with myocardial tagging was used to assess circumferential LV strain. It was shown that regional variations of LV prestretch are closely related to the regional differences of systolic LV strain amplitude and timing. The Frank-Starling mechanism is usedto explain the heterogeneity in systolic strain (i.e. systolic shortening) around the circumference of the left ventricle.

There is still a need for good approaches for determining the myocardial inotropic state.

### SUMMARY OF THE INVENTION

It is an object of embodiments of the present invention to provide good methods and systems for determining a status of the myocardial inotropic state.

It is an advantage of embodiments of the present invention that alternative devices and methods are provided for determining a myocardial inotropic state.

It is an advantage of embodiments of the present invention that methods and systems are provided that use new predictors providing a status of the myocardial inotropic state.

It is an advantage of embodiments of the present invention that predictors for a status of myocardial inotropic state are provided that can be measured in a non-invasive manner.

It is an advantage of embodiments of the present invention that the slope of the relationship between intra-ventricular stretch for different myocardial segments and the corresponding strain, e.g. obtained by myocardial deformation imaging, can be used as estimate of global LV inotropy, thus providing a non-invasive technique providing analogous results as obtained by invasive approaches.

It is an advantage of embodiments of the present invention that they use the slope of the relationship between the prestretch—strain for different myocardial segments as a measure of intrinsic LV contractility in different clinical settings. It is an advantage of embodiments of the present invention that an identifier has been found that allows to distinguish LV contractility without being influenced by changes in LV systolic function. It is an advantage of embodiments of the present invention that methods and systems are provided that use the slope of the measured relationship between prestretch-strain for different myocardial segments as distinguishing measure of contractility.

It is an advantage of embodiments of the present invention that methods and systems do not require mechanical modeling for estimating contractility but that it can be directly based on a measurement of the prestretch for different segments and its relationship to subsequent shortening.

It is an advantage of embodiments of the present invention that methods and systems are provided wherein the segmental LV prestretch is measured during passive filling, e.g. atrial contraction, and wherein this is related to the systolic strain in order to get an estimate of global chamber, e.g. LV, contractility.

The above objective is accomplished by a method and device according to the present invention.

The present invention relates to a method for determining myocardial inotropic state, whereby said determining comprises receiving a value of stretch for different myocardial segments inside the cardiac chamber, e.g. ventricle, during passive filling, e.g. atrial contraction, receiving associated values representative of total systolic strain, and using a relationship between the stretch of the myocardial segments during passive filling, e.g. atrial contraction, and the associated values representative for total systolic strain as an index of the myocardial inotropic state.

The method may be applicable for different cardiac chambers and is not limited to the left ventricle.

It is an advantage of embodiments of the present invention that the FS-mechanism is not used invasively or on the cardiomyocyctes (i.e. cell) level which are isolated from the heart and thus that the present method is in-vivo and non-invasive. It is an advantage of the present invention that the relationship is used as an index for LV inotropy and it is realized that regional differences in material properties, myofiber structure, and contractile force should not be considered, i.e. may be neglected, for a correct understanding of regional variations in myocardial function.

Using the relationship may comprise using a slope of the functional relationship between the stretch of different myocardial segments during passive filling and the associated values representative for total systolic strain as an index of the myocardial inotropic state. The functional relationship may be a linear relationship.

Using relationship may alternatively or in addition comprise using the intercept of the functional relationship between the stretch of different myocardial segments during passive filling and the associated values representative for total systolic strain as an index of the myocardial inotropic state. The functional relationship may be a linear relationship.

Values for stretch during passive filling may be values for stretch during atrial contraction.

Values for stretch during passive filling may be values for stretch during ventricular contraction applied to the atria.

The total systolic strain may be a total shortening of the myocardial segment, whereby the total shortening may be expressed as strain difference between peak late diastolic strain and end-systolic strain values.

Receiving a value of stretch and receiving a value representative of total systolic strain may comprise obtaining said value of stretch and said value representative of total systolic strain in a non-invasive way.

Obtaining said value of stretch and said value representative of total systolic strain in a non-invasive way may comprise obtaining said value of stretch and said value representative of total systolic strain based on myocardial deformation imaging.

The myocardial inotropic state may be the left ventricular isotropic state.

The method may provide distinguishing between a healthy and failing contractile state of a cardiac chamber, e.g. ventricle.

The method may comprise setting a reference point of strain curves to zero at the beginning of a P wave on an ECG, measuring a segmental prestretch of the LV as a peak positive strain during atrial contraction, and measuring systolic strain as a difference between the peak positive strain value and a peak negative systolic strain.

In embodiments of the present invention the method may comprise adapting received image data, such as e.g. tissue Doppler image (TDI), and advantageously derive strain curves for determining regional stretch—strain relationship from e.g. echocardiographic datasets.

Advantageously by using the stretch for different segments—strain relationship and more preferably by using the slope of this relationship, embodiments of the present invention may provide means to compare a response to increased preload between healthy subjects and patients with heart failure and one can test if the Frank Starling mechanism is preserved in patients with heart failure. In addition as a result embodiments of the present invention provides means to describe the differences of a contractile state between healthy and failing ventricles.

The present invention also relates to a system for determining myocardial inotropic state, the system comprising an input means for receiving a value of stretch of different myocardial segments during passive filling, e.g. atrial contraction, and receiving associated values representative of the associated total systolic strain; and a processor programmed for determining a relationship between the stretch of different myocardial segements during passive filling and the associated values representative for systolic strain, e.g. the variance amongst the stretch of the myocardial segments during passive filling and the corresponding values representative for total systolic strain, and for using the slope of this relationship as an index of the myocardial inotropic state.

The input means may be adapted for receiving a value of stretch of different myocardial segments during atrial contraction and the processing means may be programmed for determining a relationship between the stretch of the different myocardial segments during atrial contraction and the associated values.

The input means may be adapted for receiving a value of stretch of different myocardial segments during ventricular contraction applied to the atria and the processing means may be programmed for determining a relationship between the stretch of the different myocardial segments during ventricular contraction and the associated values.

The processing means may be programmed for determining a slope of a functional relationship between the stretch of the different myocardial segments during passive filling an the associated values for the systolic strain. The functional relationship may be a linear relationship. Alternatively or in addition thereto the intercept of the functional relationship with one of the axis of the graph expressing the stretch of the different myocardial segments in relation to the corresponding values for the systolic strain may be determined.

The processor furthermore may comprise a decision unit for distinguishing, based on the index, between a healthy and failing contractile state of a ventricle.

The present invention also relates to a computer program comprising computer program code means adapted to perform all the steps of a method as described above when the computer program is run on a computer.

The computer program may be embodied on a computer readable medium.

The term “data carrier” is equal to the terms “carrier medium” or “computer readable medium”, and refers to any medium that participates in providing instructions to a processor for execution. Such a medium may take many forms, including but not limited to, non-volatile media, volatile media, and transmission media. Non-volatile media include, for example, optical or magnetic disks, such as a storage device which is part of mass storage. Volatile media include dynamic memory such as RAM. Common forms of computer readable media include, for example, a floppy disk, a flexible disk, a hard disk, magnetic tape, or any other magnetic medium, a CD-ROM, any other optical medium, punch cards, paper tapes, any other physical medium with patterns of holes, a RAM, a PROM, an EPROM, a FLASH-EPROM, any other memory chip or cartridge, a carrier wave as described hereafter, or any other medium from which a computer can read. Various forms of computer readable media may be involved in carrying one or more sequences of one or more instructions to a processor for execution. For example, the instructions may initially be carried on a magnetic disk of a remote computer. The remote computer can load the instructions into its dynamic memory and send the instructions over a telephone line using a modem. A modem local to the computer system can receive the data on the telephone line and use an infrared transmitter to convert the data to an infrared signal. An infrared detector coupled to a bus can receive the data carried in the infrared signal and place the data on the bus. The bus carries data to main memory, from which a processor retrieves and executes the instructions. The instructions received by main memory may optionally be stored on a storage device either before or after execution by a processor. The instructions can also be transmitted via a carrier wave in a network, such as a LAN, a WAN or the internet. Transmission media can take the form of acoustic or light waves, such as those generated during radio wave and infrared data communications. Transmission media include coaxial cables, copper wire and fiber optics, including the wires that form a bus within a computer. The present invention thus also relates to data carriers storing a computer program product as described above and to the transmission of a computer program product according to the third aspect of the present invention over a network.

The present invention also relates to the use of a relationship between the stretch of different myocardial segments during passive filling, e.g. atrial contraction, and the associated total systolic strain as an index of the myocardial inotropic state.

The relationship may be the slope of the functional relationship between regional stretch of different myocardial segments during passive filling, e.g. atrial contraction, and the associated total systolic strain of different myocardial segments. Alternatively or in addition thereto the relationship may be the intercept of the functional relationship with one of the axis in the graph expressing the stretch of different myocardial segments in relation with the total systolic strain. The functional relationship may be a linear relationship.

The relationship may be the slope of the regional stretch-strain relationship as a non-invasive index of myocardial inotropic state. The slope may get steeper with increasing inotropy, does not change with preload induced changes of LV systolic function and flattens after the exposure to a cardiotoxic drug. Moreover, the presence of a regional stretch—strain relationship in an individual ventricle shows that a major part of intraventricular variability of systolic strain can be explained by segmental differences in passive stretch during atrial contraction.

Preferred embodiments of the invention provide a predictor which provides a status of the myocardial inotropic state, whereby said predictor is preferably based on a slope of this intra-ventricular stretch-strain relationship, whereby said relationship advantageously can be measured by non-invasive methods, such as e.g. myocardial deformation imaging and used as an estimate of global LV inotropy in analogy to the invasive approaches.

In some embodiments the presence of a regional stretch—strain relationship in an individual ventricle may show that a major part of intraventricular variability of systolic strain can be explained by segmental differences in passive stretch during atrial contraction as a direct consequence of the Frank-Starling mechanism. The slope of this relationship gets steeper with increasing inotropy, does not change with preload induced changes of LV systolic function and flattens after the exposure to a cardiotoxic drug, suggesting that it could serve as a non-invasive index of myocardial contractility.

In some embodiments of the present invention a hypothesis was tested, namely that the non-invasively constructed slope of the relationship between LV regional systolic strain and stretch during atrial contraction represents LV inotropic state. LV systolic response to a changing preload depends on its inotropic state. Changing the preload has allowed constructing the slope of the end-systolic pressure-volume relationship that is used as an invasive measurement of left ventricular (LV) inotropy. According to an aspect of the present invention it has been assumed that the slope of the relationship between regional systolic LV strain (total_S) and stretch during atrial contraction (preS) depends on the LV inotropic state as well and can thus be used as a LV inotropy index.

In preferred embodiments strain curves (e.g. obtained by applying Tissue Doppler methodology) may be extracted from healthy individuals to determine the normal stretch-strain relationship at rest, during a low dose dobutamine (LD) challenge and/or during a passive leg-lift (LL).

Embodiments of the invention advantageously provide means to obtain a regional stretch—strain relationship per ventricle using myocardial deformation imaging, whereby said stretch—strain relationship advantageously can be regarded as a non-invasive application of the FS relationship on regional level. Moreover, using a method according to embodiments of the invention, it has been found that at rest systolic strain can be dependent from the amount of late diastolic stretch in healthy subjects and in patients with mild to moderate heart failure. The slopes of the stretch—strain relation appeared to remain the same in those groups. However, systolic LV response to the increased venous return was blunted in the failing ventricles due to the failure to increase the passive stretch with increasing preload. Moreover, we could presume that there was increased contractility in young healthy subjects due to the increased preload, whereas in older subjects this contractility increase was absent and augmentation of systolic function during increased preload was purely Frank-Starling mechanism related.

Embodiments of the invention advantageously provide a non-invasively constructed slope of the relationship between LV regional systolic strain and stretch during atrial contraction represents LV inotropic state.

The method was also applied in healthy volunteers during low dose dobutamine (LD), during passing leg lifting (LL) and in patients with breast cancer before and after chemotherapy (FU) with anthracyclines. PreS and total_S correlated closely in all subjects (r=0.82). Total_S values increased (p<0.05) with LD (−20.44±3.89% vs. −24.24±5.55%) and LL (−19.65±3.77% vs. −24.05±3.67%), whereas preS increased only with LL (5.96±1.72% vs. 8.61±2.18%), but not with LD (6.83±2.34% vs. 7.29±2.24%). No changes of total_S or preS were observed after the exposure to chemotherapy (−21.23±2.93% vs. −21.49±2.89% and 8.11±1.03% vs. 8.59±1.73%, respectively). The slope of stretch-strain relationship got steeper with LD (−1.47±0.36 vs. −2.34±0.36, p<0.05), declined after the chemotherapy (−1.68±0.15 to −0.86±0.23, p<0.05) and did not change with LL (−1.39±0.57 vs. −1.51±0.38, ns).

In preferred embodiments of the invention a segmental systolic LV strain is related to segmental stretch of myocardium during atrial contraction to construct a regional stretch-strain relationship. Moreover, in preferred embodiments the slope of this relationship gets significantly steeper in response to a dobutamine challenge and does not change with preload induced increase of LV function. Thus, in preferred embodiments, the slope may be regarded as an index of LV inotropic state. The proposed stretch-strain relationship could potentially serve in clinical routine, when a detection of deteriorating intrinsic LV function is important.

It is an advantage of at least some embodiments of the present invention that a particular way of extracting regional strain curves is provided. The method comprises setting a reference point of the strain curves to zero at the beginning of the P wave on an ECG and measuring the segmental prestretch of the LV as a peak positive strain during atrial contraction. The method may also comprise subsequently measuring systolic strain as a difference between the positive strain peak value and the peak negative systolic strain.

It is an advantage of at least some embodiments of the present invention that use can be made of conventional strain curves, whereby LV prestretch can be calculated as a difference between the strain value measured at the beginning of atrial contraction and the strain value at end-diastole.

Embodiments of the present invention thus may provide a method using the behavior of the stretch-strain relationship as an index or predictor for an increasing in LV inotropy. However, theoretically it can be expected that a reduced LV inotropic state results in a shallower slope of this relationship.

Moreover, the proposed stretch-strain relationship used in preferred embodiments of the invention can advantageously potentially serve in a clinical routine, when a detection of deteriorating intrinsic LV function is important. It could possibly be applied for the follow up of patients with mitral or aortic valve regurgitation as in those patients early detection of decreasing myocardial inotropy is crucial for the correct timing of surgical intervention. Moreover, it might be a beneficial parameter to monitor the treatment of heart failure patients, to detect cardiotoxicity in patients undergoing chemotherapy or to differentiate physiological forms of LV hypertrophy from the pathological ones. All of those potential implications of LV stretch-strain relationship remain the topics for future studies. Moreover, the presence of a regional stretch-strain relationship in an individual ventricle shows that a major part of intraventricular variability of systolic strain can be explained by segmental differences in passive stretch during atrial contraction as a direct consequence of the Frank-Starling mechanism. The slope of this relationship gets steeper with increasing inotropy and does not change with preload induced changes of LV systolic function, suggesting that the slope, according to preferred embodiments of the invention, may be regarded as a non-invasive index of myocardial contractility. In addition, such an index might be applied for the early detection of deteriorating myocardial intrinsic inotropy in various cardiac pathologies.

Particular and preferred aspects of the invention are set out in the accompanying independent and dependent claims. Features from the dependent claims may be combined with features of the independent claims and with features of other dependent claims as appropriate and not merely as explicitly set out in the claims.

These and other aspects of the invention will be apparent from and elucidated with reference to the embodiment(s) described hereinafter.

### BRIEF DESCRIPTION OF THE DRAWINGS

Further features of the present invention will become apparent from the examples and figures, wherein:

FIG. 1 shows the adapted myocardial deformation curves and measurements, more specifically schematic representation (a) and an example (b) of adapted myocardial deformation curves and measurements performed according to embodiments of the present invention. In FIGS. 1 (a) and (b) preS relates to the stretch of myocardial segment during the atrial contraction, total_S relates to the total systolic strain. MVC relates to mitral valve closure, AVO to aortic valve opening, AVC to aortic valve closure, MVO to mitral valve opening, AC to atrial contraction, IVC to isovolumic contraction period, IVR to isovolumic relaxation, and E to early diastole.

FIG. 2 shows the normal stretch-strain relationship and regression equation at rest, according to an embodiment of the present invention. Each colored line represents the preS-total_S relationship in individual subject, where the results from each LV myocardial segment were used to draw a regression line representing the relationship. The black dashed line represents the mean regression line with 95% CI of all 16 subjects. In FIG. 2 preS relates to the stretch of myocardial segment during the atrial contraction and total_S to the total systolic strain.

FIG. 3 shows an example of stretch-strain relationship changes in response to increased and decreased inotropy. Moreover FIG. 3 illustrates stretch-strain relationships and regression equations a) at rest (blue line) and during the low dose dobutamine challenge (green line) in one of the healthy study subjects, and b) at the baseline (blue line) and after 3 cycles of chemotherapy with anthracycline (green line) in one of the patients with breast cancer. PreS relates to the stretch of myocardial segment during the atrial contraction, total_S to the total systolic strain, BL relates to rest and LD to low dose dobutamine challenge.

FIG. 4 shows changes of stretch-strain relationship in response to increased inotropy, increased preload and decreased inotropy. Moreover FIG. 4 illustrates stretch-strain relationships and regression equations a) at rest (blue line) and during the low dose dobutamine challenge (red line), b) at rest (blue line) and during the passive leg lift (red line) and c) at baseline (blue line) and after 3 cycles of treatment with anthracycline (red line). The regression lines are obtained by averaging slopes and intercepts obtained per patient. For graphical display, they are shown together with the mean values that are represented by dots. PreS relates to the stretch of myocardial segment during the atrial contraction, whereas total_S relates to the total systolic strain.

FIG. 5 shows individual changes of the slope of stretch strain relationship with increased and decreased inotropy. Moreover FIG. 5 illustrates changes of the slope of stretch-strain relationship in individual patients (represented by colored lines) a) during the low dose dobutamine challenge and b) after 3 cycles of chemotherapy.

FIG. 6 shows the relationship between Δstretch and Δstrain in the passive leg lift group. Δ_PreS relates to the change of segmental stretch of myocardium during the atrial contraction with passive leg lift, whereas Δ_total_S relates to the change of total segmental systolic strain with passive leg lift. Dashed line represents 95% confidence interval of the regression line.

The drawings are only schematic and are non-limiting. In the drawings, the size of some of the elements may be exaggerated and not drawn on scale for illustrative purposes.

Any reference signs in the claims shall not be construed as limiting the scope.

In the different drawings, the same reference signs refer to the same or analogous elements.

### ABBREVIATIONS

LV—left ventricle;
ESPVR—end-systolic pressure volume relationship;
PRSW—preload recruitable stroke work;
TDI—tissue Doppler imaging;
FR—frame rate;
BL—resting state;
FU—follow-up;
LD—low dose dobutamine challenge;
LL—passive leg-lift;
LV EDV—left ventricular end-diastolic volume;
HR—heart rate;
LV ESV—left ventricular end-systolic volume;
LV EF—left ventricular ejection fraction;
LV SI—left ventricular sphericity index;
LV WS—left ventricular wall stress;
preS—left ventricular stretch during atrial contraction;
total_S—left ventricular systolic shortening;
LV SV—left ventricular stroke volume;
Δ_preS—change of left ventricular stretch during atrial contraction with leg-lift;
Δ_total_S—change of left ventricular strain with leg-lift;
P-V—pressure-volume;
MRI—magnetic resonance imaging;
SPECT—single photon emission tomography;
LBBB—left bundle branch block.

### DETAILED DESCRIPTION OF ILLUSTRATIVE EMBODIMENTS

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes. The dimensions and the relative dimensions do not correspond to actual reductions to practice of the invention.

Furthermore, the terms first, second and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

Moreover, the terms top, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

Where in embodiments of the present invention reference is made to “a value of total systolic strain”, reference is made to a value of shortening response or a value of segmental shortening. Preferably the term relates to a total value of systolic shortening.

Contraction or contractility is a clinically useful term, that can provide a distinguishing measure of a better-performing heart from a poorly performing one. Contractility can be a measure for intrinsic contractile performance independent from external factors or can provide a measure of the intrinsic ability of a heart muscle to generate force at certain rate and time (controlled for loading conditions). Moreover, contractility can relate to the property of cardiac muscle that determines its ability to shorten independent of preload and afterload.

In a first aspect, the present invention relates to a method for determining myocardial inotropic state, e.g. the left ventricular isotropic state. For example, a method according to embodiments of the present invention may provide distinguishing between a healthy and failing contractile state of a ventricle.

This determining comprises receiving a value of stretch of different myocardial segments during passive filling, e.g. atrial contraction or ventricular contraction when applied to the atria. This receiving may comprise obtaining the value of stretch for different myocardial segements in a non-invasive way, e.g. obtaining the value of stretch for different myocardial segements based on myocardial deformation imaging.

The determining further comprises receiving associated, e.g. corresponding, values representative of total systolic strain. This receiving may comprise obtaining the associated values representative of total systolic strain in a non-invasive way, e.g. obtaining the associated values representative of total systolic strain based on myocardial deformation imaging. The total systolic strain may be a total shortening of the myocardial segment, in which this total shortening is expressed as strain difference between peak late diastolic strain and end-systolic strain values.

In some embodiments, strain imaging may be used, whereby strain imaging can provide regional detection of myocardial contraction. It enables clinicians to determine and receive velocity gradients along the ultrasound beam, thereby helping users analyze tissue contraction and regional myocardial function. In embodiments of the invention, strain and strain rate imaging can be used to evaluate ischemic heart disease. Strain Imaging preferably can measure a percent of regional deformation of the myocardium, while Strain Rate Imaging can measure the speed of deformation. The majority of strain rate changes are too fast to be detected by the human eye in real time. With the application of post-processing tools, the comparison of strain or strain rate traces from different myocardial regions allows detailed insight into regional mechanical function. As an added benefit, the analysis of strain and strain rate information is minimally affected by motion or tethering effects of the heart.

In other embodiments an echocardiographic examination may be performed with e.g. a GE Vingmed Vivid E9 scanner (GE Vingmed Ultrasound, Horten, Norway), preferably equipped with 2.5 MHz M5S transducer. Preferably B-mode acquisitions of 4 chamber and 2 chamber views, pulsed wave Doppler recordings of the LV outflow tract and the mitral valve inflow, as well as narrow sector TDI (FR 180-210 Hz) images of all 6 LV walls are used.

In other embodiments B-mode acquisitions of 4 chamber, 2 chamber and apical long axis views with underlying TDI (FR 100-120 Hz) can be acquired at rest (BL) and at the state of increased myocardial inotropy induced by a low dose, e.g. 10 μg/kg/min dobutamine challenge (LD). Peripheral brachial artery blood pressure was preferably measured at both stages e.g. by using an electronic sphygmomanometer.

The determining further comprises using a relationship between the values of stretch for different myocardial segments during passive filling and the associated values representative for total systolic strain as an index of the myocardial inotropic state. In embodiments according to the present invention, this relationship may be a slope in a functional relationship between the stretch for different myocardial segments during passive filling and the total systolic strain. In embodiments of the invention, such a slope may be used as an estimate of myocardial inotropic state. The relationship may for example be determined by determining the slope on a functional relationship obtained by setting out on one axis of a graph the stretch for different myocardial segments during passive filling and on the other axis of the graph the associated, e.g. corresponding, values representative for total systolic strain.

Furthermore, the method may comprise setting a reference point of strain curves to zero at the beginning of a P wave on an ECG, measuring a segmental prestretch of the LV as a peak positive strain during atrial contraction, and measuring systolic strain as a difference between the peak positive strain value and a peak negative systolic strain.

In a second aspect, the present invention relates to a system for determining myocardial inotropic state.

Such system comprises an input means for receiving a value of stretch of different myocardial segments during passive filling, e.g. atrial contraction or ventricular contraction applied to the atria, and receiving associated, e.g. corresponding, values representative of total systolic strain. For example, in a system according to embodiments of the present invention, the input means may comprise means for receiving the value of stretch for different myocardial segments and the associated values representative of systolic strain as analog or digital electrical signals, e.g. an analog to digital converter connected to at least one signal line or a digital communication bus interface. The input means may also comprise means for receiving these values on a data carrier, e.g. a magnetic storage medium reader or a removable memory connector, e.g. a flash memory connector. However, the input means may also comprise a measurement device, e.g. a echocardiography unit, such as an echocardiography unit adapted for measuring global or regional myocardial function, more particularly adapted for measuring data providing values of stretch for different myocardial segments and the associated values representative of systolic strain. Measuring global or regional myocardial function may comprise myocardial deformation imaging.

The system further comprises a processing means, e.g. a processor or processing unit, programmed for determining a relationship between the stretch of different myocardial segments during passive filling, e.g. atrial contraction or ventricular contraction applied to the atria, and the associated values representative for total systolic strain and for using the relationship as an index of the myocardial inotropic state. The processing means may be programmed for determining a slope and or an intercept of the functional relationship of the stretch of the different myocardial segment during passive filling and the total systolic strain as this relationship. The processing means may furthermore comprise a decision unit for distinguishing, based on the index, between a healthy and failing contractile state of a ventricle.

In a further aspect, the invention also relates to a computer program comprising computer program code means adapted to perform all the steps of a method according to the first aspect of the present invention when the computer program is run on a computer, e.g. when the computer program is executing on a processing means, for example in a device according to the second aspect of the present invention.

Embodiments of the present invention may thus also relate to computer-implemented methods for performing at least part of a method for determining myocardial inotropic state according to the first aspect of the invention. The methods may be implemented in a computing system. They may be implemented as software, as hardware or as a combination thereof. Such methods may be adapted for being performed on computer in an automated and/or automatic way. In case of implementation or partly implementation as software, such software may be adapted to run on suitable computer or computer platform, based on one or more processors. The software may be adapted for use with any suitable operating system such as for example a Windows operating system or Linux operating system. The computing means may comprise a processing means or processor for processing data. According to some embodiments, the processing means or processor may be adapted for determining myocardial inotropic state according to any of the methods as described above. Besides a processor, the computing system furthermore may comprise a memory system including for example ROM or RAM, an output system such as for example a CD-rom or DVD drive or means for outputting information over a network. Conventional computer components such as for example a keyboard, display, pointing device, input and output ports, etc also may be included. Data transport may be provided based on data busses. The memory of the computing system may comprise a set of instructions, which, when implemented on the computing system, result in implementation of part or all of the standard steps of the methods as set out above and optionally of the optional steps as set out above. The obtained results may be outputted through an output means such as for example a plotter, printer, display or as output data in electronic format.

The present invention also relates to a computer program according to embodiments of the present invention embodied on a computer readable medium. Thus, embodiments of the present invention may encompass computer program products embodied in a carrier medium carrying machine readable code for execution on a computing device, the computer program products as such as well as the data carrier such as dvd or cd-rom or memory device. Aspects of embodiments furthermore encompass the transmitting of a computer program product over a network, such as for example a local network or a wide area network, as well as the transmission signals corresponding therewith.

In a yet further aspect, the present invention relates to the use of a relationship between values for the stretch of different myocardial segments during passive filling and the total systolic strain as an index of the myocardial inotropic state. This relationship may be a slope of the functional relationship between the stretch of different myocardial segments during passive filling and the total systolic strain.

By way of illustration, embodiments of the present invention not being limited thereto, a number of examples and studies are reported, illustrating standard and optional features of embodiments of the present invention.

### Study Population

Embodiments of the methods and devices are elaborated on a study population in this application. However, the various embodiments are not limited to any particular point of reference or means of determining the point of reference. Furthermore, the various embodiments will be illustrated with reference to application to the left ventricle, but it will be clear to the skilled person that the method is equally applicable to another cardiac chamber, embodiments thus not being limited to the left ventricle.

TABLE 1
Exclusion criteria
Exclusion criteria

At least one of the following
significant (≧50%) coronary artery stenosis on angiography in the
previous 4 years
signs of relevant ischemic heart disease on perfusion and delayed
enhancement MRI or SPECT,
previous hospital admission with signs suggestive of myocardial ischemia/
elevated cardiac enzymes,
signs of myocardial infarction, arrhythmias, LV hypertrophy or conduction
disturbancies (AV block, LBB) on the ECG
systolic or diastolic LV dysfunction or no signs of structural heart disease
on the baseline echocardiographic examination

Thirty five healthy individuals and 7 patients with breast cancer undergoing chemotherapy with cardiotoxic anthracycline were recruited to the study. All study participants were free from cardiovascular disease (table 1). The baseline echocardiographic examination in those individuals showed a sinus rhythm, normal LV systolic and diastolic function and ruled out any structural heart disease. All study subjects signed an informed consent before inclusion. The study complied with the Declaration of Helsinki and the local ethical committee approved the study protocol. In Table 1 LV relates to left ventricle, LV EF to left ventricular ejection fraction, MRI to magnetic resonance imaging, SPECT to single photon emission tomography, and LBBB to left bundle branch block.

The study population of healthy subjects was split in three groups. 1) The normal stretch-strain relationship, according to embodiments of the present invention, was defined in 19 individuals. 2) To test the effect of increased inotropy on the slope of the stretch-strain relationship, according to embodiments of the present invention, LV inotropy was preferably modulated pharmacologically in a subset of 8 individuals from this first group. 3) The third group consisted of the remaining 16 subjects in whom an acute increase of LV preload was induced by passive leg-lifting to test its effect on the stretch-strain relationship, according to embodiments of the present invention. Finally, the effect of the decreasing contractility on the slope of stretch-strain relationship was tested in patients with breast cancer before and after 3 cycles of standard chemotherapy with anthracycline.

### Study Protocol

An echocardiographic examination was performed with a GE Vingmed Vivid 7 or E9 scanners (GE Vingmed Ultrasound, Horten, Norway), equipped with 2.5 MHz M3S and M5S transducers. B-mode acquisitions of 4 chamber and 2 chamber views, pulsed wave Doppler recordings of the LV outflow tract and the mitral valve inflow were acquired. In addition, the sector size was reduced in order to obtain narrow sector tissue Doppler imaging (TDI) acquisitions (FR 180-210 Hz) of properly aligned LV walls (inferoseptal, anterolateral, anterior, inferior, inferolateral and anteroseptal respectively). This protocol was followed in the first group of healthy individuals and in the breast cancer patients, where it was used both at baseline (BL) (i.e. within 4 weeks before the start of a standard chemotherapy protocol with anthracycline) and at follow-up (FU) (i.e. within 7 to 14 days after the third chemotherapy cycle).

In the second group of healthy subjects B-mode acquisitions of 4 chamber, 2 chamber and apical long axis views with underlying TDI (FR 100-120 Hz) were acquired at rest (BL) and at the state of increased myocardial inotropy induced by a low dose (10 μg/kg/min) dobutamine challenge (LD). Hereto, dobutamine infusion was started after acquisition of the baseline images and continued for 3 minutes. After 3 minutes the LD stage images were recorded and the dobutamine infusion was stopped. Peripheral brachial artery blood pressure was measured at both stages using an electronic sphygmomanometer.

To investigate the influence of acute increase in LV preload, narrow sector TDI (FR 180-210 Hz) of the inferoseptal wall, pulsed wave Doppler of the mitral valve inflow and an additional apical tri-plane image of the LV were continuously recorded at rest and during subsequent passive leg-lifts (LL) in the third study group of healthy subjects. Hereto, both legs of the supine individual were lifted to an angle of approximately 30° from a horizontal position and kept in that position for 30 s while continuously recording echocardiographic data. The 30 s time span was chosen as the preload effect of this maneuver is acute and rather short lived, as described in Monnet X et al. in Applied Physiology in Intensive Care Medicine 2^{nd} edition edited by Springer-Verlag Berlin Heidelberg 2009, 185-190 and an acute increase of LV end-diastolic volume (LV EDV) as well as typical changes of mitral inflow without a change in heart rate (HR) occur already after 15 s, as described by Downes T R et al. in The American journal of cardiology 1990 (65) 377-82. After the legs are returned to the horizontal position all preload induced changes are known to disappear completely, as described in Monnet X et al. in Applied Physiology in Intensive Care Medicine 2^{nd} edition edited by Springer-Verlag Berlin Heidelberg 2009, 185-190. Therefore, the passive leg lifts could be performed repeatedly and separate continuous acquisitions of inferoseptal wall, mitral inflow and LV triplane view could be obtained.

The LV EDV measured from the triplane recordings was used to define the cardiac cycle with the largest preload (i.e. end-diastolic volume). Peripheral blood pressure was continuously monitored during the passive LL with a commercially available Finometer™ system.

### Data Analysis

Conventional echocardiographic data were analyzed using commercially available software (GE, Echopac version 110.1.2). LV end-diastolic (LV EDV) and end-systolic (LV ESV) volumes, as well as LV ejection fraction (LV EF) were measured from apical 4 and 2 chamber views using Simpson's biplane method. In subjects that underwent a leg-lift test, LV EDV, ESV and EF were calculated continuously from the triplane LV volume acquisition.

The LV sphericity index (LV SI) at end-diastole, giving an estimate of global LV shape, was calculated by dividing the LV EDV by the volume of a sphere with the same long axis dimension. The latter parameter was calculated as 4/3×π×(long axis diameter at end-diastole/2)³, as described in Kaku K. et al. in Journal of the American Society of Echocardiography: official publication of the American Society of Echocardiography 2011 (24) 541-547. Global LV end-systolic wall stress (WS) was calculated by the formula: WS=(p*r)/2 h, where p is the peripheral systolic blood pressure, r is the effective radius of the LV (calculated as 3/(¾ LV ESV×π)) and h is the LV wall thickness, measured as an average of mid segments of septal and lateral LV walls from parasternal long axis images.

Peak E-wave, peak-A wave velocities and E wave deceleration time can be measured from the pulsed wave Doppler recordings of the mitral inflow.

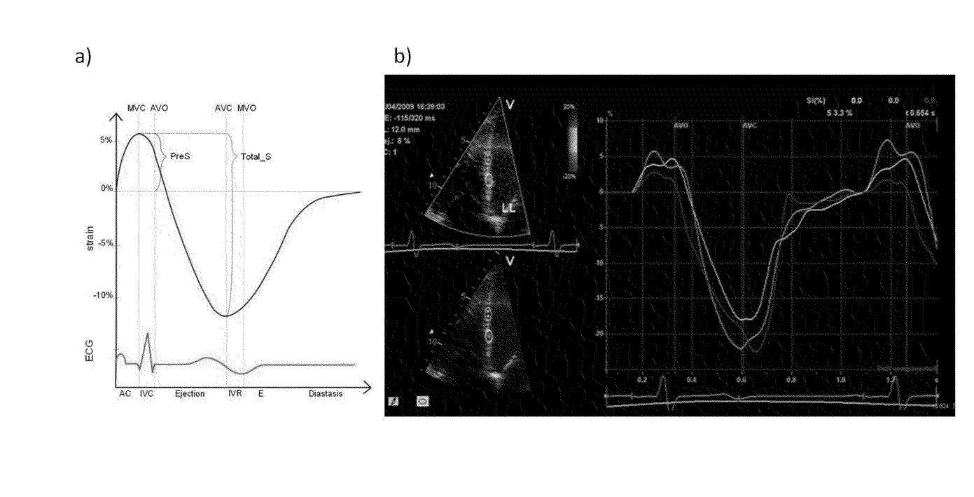

The same software, Echopac, was used for myocardial deformation analysis. Hereto, the onset of the P wave on the ECG, indicating the beginning of atrial contraction, instead of the start of the QRS complex was chosen as a zero reference point for deformation. The timing of mitral valve closure, aortic valve opening, aortic valve closure and mitral valve opening were measured from the Doppler recordings. Three samples (size 12×6 mm) were distributed equally from the base to the apex of each LV wall and manually tracked through the cardiac cycle to ensure their position within the myocardial segment. Segments were the tracking was failing were excluded from further analysis. From the obtained segmental myocardial deformation curves lengthening or stretch (preS) of the LV during atrial contraction was measured as the peak positive strain (%) during the atrial contraction. The total systolic strain (total_S) was defined as a total shortening (%) of the segment (i.e., strain difference between the peak late diastolic strain and end-systolic strain values) (see e.g. FIG. 1).

In patients that received a low dose dobutamine challenge all the parameters were calculated at BL and LD stages. In case the passive leg-lift test was performed, all the parameters were measured at BL and during the peak preload increase, which was defined as the cardiac cycle with the highest increase of LVEDV during the passive LL. In this way we made sure that an acute LV response to the preload challenge was measured and that no reflex-mediated changes of inotropy were occurring. Finally, in the patients undergoing chemotherapy the same parameters were calculated at the BL and at the FU stages.

### Stretch-Strain Relationship

To obtain the stretch-strain relationship, according to embodiments of the present invention, within a ventricle linear regression lines were estimated through 18 segmental preS and total_S values in every individual. For the subjects, that underwent passive LL regression lines were drawn through 3 segmental values extracted from basal, mid and apical levels of the inferoseptal wall. The obtained intercepts and slopes were averaged per group and per stage to represent the mean relation.

In order to test reproducibility of the regression equations 10 randomly chosen rest studies from the first subset of healthy subjects were reanalyzed by the same observer blinded to the initial results.

In embodiments of the present invention statistical analysis may be performed with SPSS version 18.0 (SPSS, Inc, Chicago, Ill.). Values are expressed as mean±standard deviation. Variables were checked to be normally distributed (visually from the appearance of the histograms) and to have equal variances (Levene's test of homogeneity). Independent samples t-test was performed to detect significant differences between the groups. Significant changes of parameters at different stages were determined by paired samples t-test. A p-value below 0.05 was considered statistically significant. Intraobserver variability was calculated as a mean error between two repeated measurements. By study design, it was not possible to analyze the echocardiographic images blinded with regard to the inotropic or preload modulation.

The demographic information and echocardiographic characteristics of the study population are summarized in Table 2. A total of 27 healthy individuals and 7 patients with breast cancer undergoing treatment with cardiotoxic anthracycline were included. Three individuals from the first subgroup and 5 from the third subgroup of the healthy study population were excluded due to suboptimal TDI image quality.

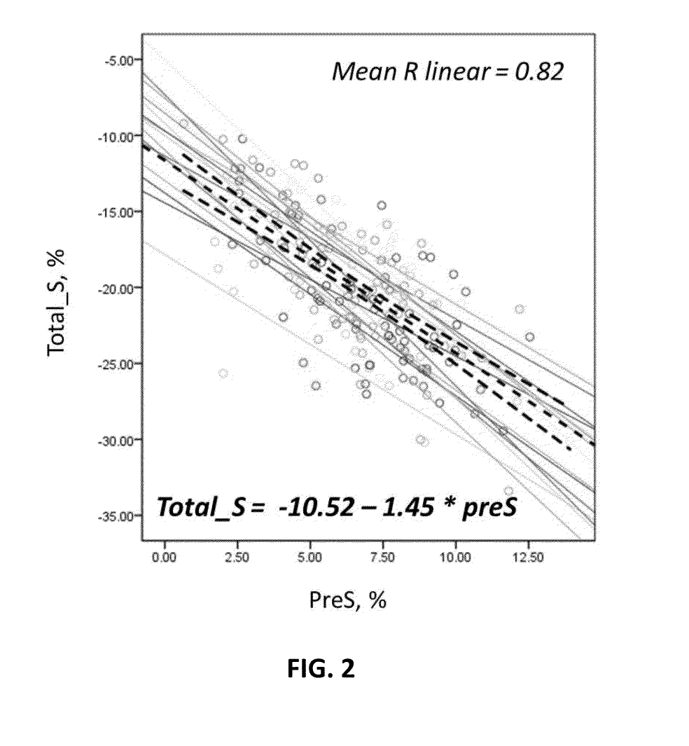

In the first subset of subjects (n=16) mean segmental preS was 6.7±2.49%, and mean segmental total_S was −20.15±4.49%. As shown in FIG. 2, those two parameters correlated closely amongst the LV segments in every patient (r=0.82; range from 0.69 to 0.95). The mean intercept of the regression lines was −10.52±3.14 (range from −5.05 to −17.8) with a mean slope of −1.45±0.28 (range from −1.01 to −1.9). The same observer could reproduce individual intercepts with a mean error of 19.7% and slopes—with a mean error of 12%.

TABLE 2
General characteristics and echocardiographic parameters of the study population:

| | First group* | | | | | | |
|---|---|---|---|---|---|---|---|
| | (n = 16) | | | | | | |
| | | Dobutamine group | | | | | |
| | All subjects of | (a subset (n = 8) | | Passive leg-lift group | | Anthracycline group^{†} | |
| | the first group | of the first group) | | (n = 11) | | (n = 7) | |
| | (n = 16) | BL | LD | BL | LL | BL | FU |
| Mean age | 56.1 ± 13.6 | 58.5 ± 10.8 | | 52.91 ± 3.33 | | 71.6 ± 3.1 | |
| (years) | | | | | | | |
| Male/ | 9/7 | 6/2 | | 6/5 | | 0/8 | |
| female | | | | | | | |
| BMI | 26.06 ± 3.83 | 24.37 ± 3.58 | | 24.6 ± 1.75 | | 25.95 ± 4.77 | |
| (kg/m²) | | | | | | | |
| Heart rate | 59.57 ± 9.26 | 60.39 ± 8.71 | 67.98 ± 10.89^{‡} | 65.64 ± 9.1 | 67.64 ± 7.89 | 65 ± 8.37 | 70.67 ± 10.59^{‡} |
| (bpm) | | | | | | | |
| Syst. ABP | 135.3 ± 17.14 | 135 ± 13.88 | 149 ± 17.58^{‡} | 138.94 ± 19.17 | 144.01 ± 17.65 | 138 ± 8.98 | 142.86 ± 21.15 |
| (mmHg) | | | | | | | |
| Diast. | 81.1 ± 10.03 | 83 ± 9.02 | 75.2 ± 8.44^{‡} | 71.74 ± 27.53 | 77.53 ± 10.71 | 79.43 ± 6.39 | 75.86 ± 14.97 |
| ABP | | | | | | | |
| (mmHg) | | | | | | | |
| LV EDV (ml) | 91.44 ± 21.71 | 88.63 ± 19.12 | 88.13 ± 17.27 | 107.14 ± 14.97^{§} | 120.57 ± 16.12^{§‡} | 60.57 ± 18.27 | 67.71 ± 19.68^{‡} |
| LV ESV (ml) | 36.19 ± 10.3 | 35.75 ± 9.82 | 30.88 ± 10.15^{‡} | 45.29 ± 11.08^{§} | 46.29 ± 10.49^{§} | 21.43 ± 9.99 | 25.29 ± 10.23 |
| LV EF (%) | 60.56 ± 6 | 60.13 ± 3.94 | 65.75 ± 4.98^{‡} | 57.43 ± 4.65^{§} | 62.57 ± 4.58^{§‡} | 69.43 ± 9.03 | 64.21 ± 6.94^{‡} |
| Sphericity | 0.33 ± 0.07 | 0.32 ± 0.04 | 0.33 ± 0.03 | 0.3 ± 0.04 | 0.31 ± 0.04 | 0.31 ± 0.13 | 0.31 ± 0.09 |
| index | | | | | | | |
| LV WS | 214.85 ± 47.42 | 225.97 ± 52.5 | 215.55 ± 46.83 | 226.06 ± 31.14 | 231.87 ± 23.97 | 193.56 ± 45.73 | 211.58 ± 52.16 |
| (mmHg) | | | | | | | |
| E velocity | 0.69 ± 0.12 | 0.7 ± 0.13 | — | 0.67 ± 0.1 | 0.78 ± 0.86^{‡} | 0.67 ± 0.11 | 0.74 ± 0.0.21 |
| (cm/s) | | | | | | | |
| A velocity | 0.68 ± 0.18 | 0.67 ± 0.2 | — | 0.53 ± 0.13 | 0.59 ± 0.11^{‡} | 0.76 ± 0.22 | 0.89 ± 0.31^{‡} |
| (cm/s) | | | | | | | |
| E/A ratio | 1.12 ± 0.35 | 1.13 ± 0.38 | — | 1.33 ± 0.36 | 1.38 ± 0.29 | 0.94 ± 0.28 | 0.88 ± 0.27 |
| E wave | 225.61 ± 50.60 | 203.49 ± 45.72 | — | 212.36 ± 15.45 | 201.73 ± 35.59 | 190.71 ± 48.46 | 199.71 ± 43.58 |
| DecT | | | | | | | |
| (ms) | | | | | | | |

*individuals used to define normal prestretch-strain relationship,
^{†}patients with breast cancer undergoing treatment with cardiotoxic anthracycline,
^{‡}p < 0.05 against the baseline of the same group,
^{§}calculated from triplane acquisitions.
BL—baseline,
LD—low dose dobutamine,
LL—passive leg-lift,
FU—follow-up after 3 cycles of chemotherapy,
BMI—body mass index,
ABP—arterial blood pressure,
LV EDV—left ventricular end-diastolic volume,
LV ESV—left ventricular end-systolic volume,
LV SV—left ventricular stroke volume,
LV EF—left ventricular ejection fraction,
DecT—deceleration time,
LV WS—left ventricular wall stress

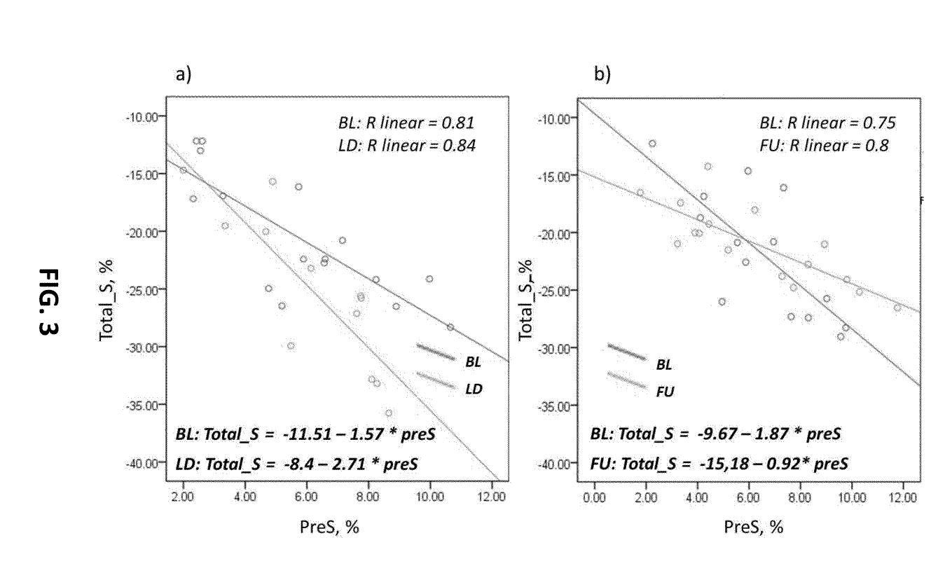

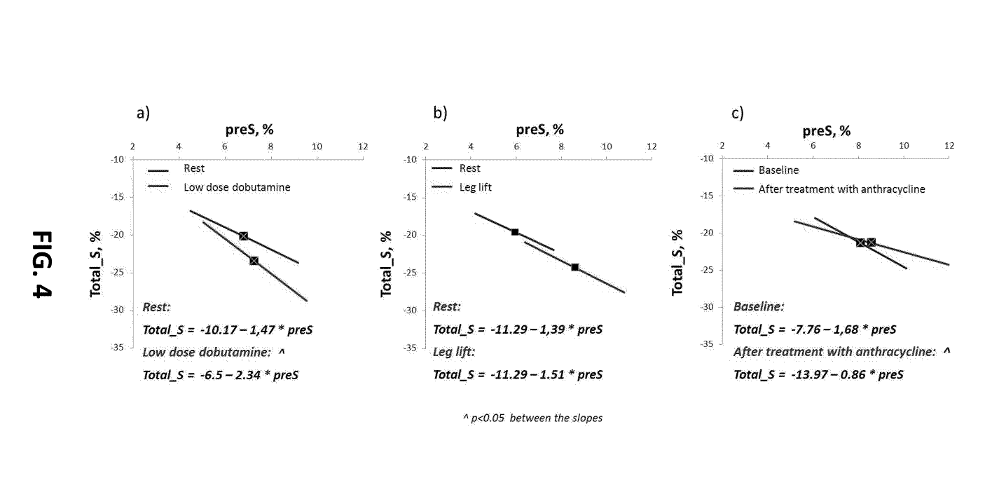

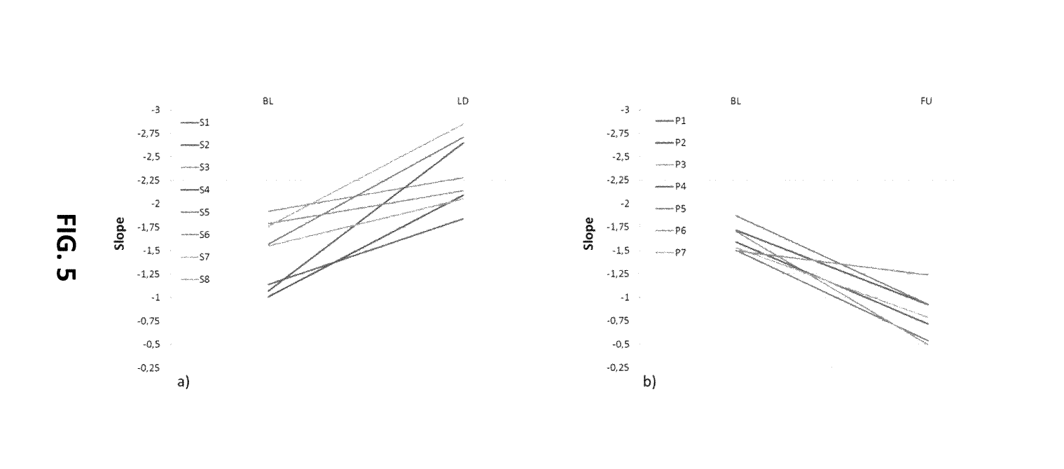

Low dose dobutamine infusion resulted in a decrease of LVESV, an increase of LV stroke volume (SV), and an increase of LVEF. LV WS and SI, on the other hand, did not show any significant changes in response to dobutamine (table 2). Segmental total_S increased significantly (−20.44±3.89% vs. −24.24±5.55%, p<0.05), while segmental preS did not change (6.83±2.34% vs. 7.29±2.24%, ns.) with dobutamine challenge (table 3). A typical example of stretch-strain relationship response to low dose dobutamine is given in FIG. 3a. The mean slope of the preS-total_S regression lines increased significantly from −1.47±0.36 to −2.34±0.36 (p<0.05) and the mean intercept decreased from −10.17±2.39 to −6.5±4.73 (p<0.05) (table 3, FIG. 4a). This response of the stretch-strain relationship was seen in every individual (FIG. 5a).

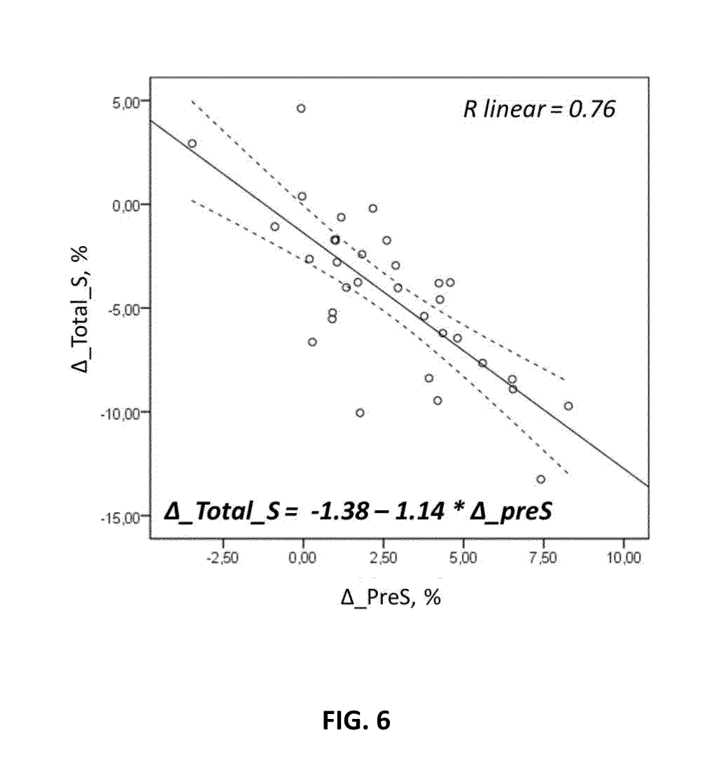

During the passive leg-lift LVEDV, LV SV, LV EF, E-wave, and A-wave velocities increased significantly (see e.g. Table 2), while LV SI, blood pressure, global LV WS and heart rate did not change from baseline (table 2). Both preS and total_S increased significantly with LL (5.96±1.72% vs. 8.61±2.18%, p<0.05 and −19.65±3.77% vs. −24.05±3.67%, p<0.05) (table 3). No change of the mean slopes and intercepts of the regression lines between preS and total_S were observed (−1.39±0.57 vs. 1.51±0.38 and −11.29±2.34 vs. −11.29±4.04, respectively) (table 3, FIG. 4b). Change of preS (Δ_preS) during the LL correlated significantly (r=0.76) with the change of total_S (Δ_total_S) (FIG. 6).

All the breast cancer patients had normal LV systolic and diastolic function at baseline. After the treatment with anthracycline a significant increase of LVEDV and a decrease of LV EF was observed (table 2), whereas LV SI and global LV WS did not change. Similarly, total_S and preS did not change significantly from baseline (−21.23±2.93% vs. −21.49±2.89% and 8.11±1.03% vs. 8.59±1.73%, p<0.05, respectively) (see e.g. table 3). In Table 3 * relates to individuals used to define normal prestretch-strain relationship, † to patients with breast cancer undergoing treatment with cardiotoxic anthracycline, ‡ to p<0.05 against the baseline of the same group. Moreover BL relates to baseline, LD to low dose dobutamine, LL to passive leg-lift, FU to follow-up after 3 cycles of chemotherapy, preS to stretch of myocardial segment during the atrial contraction, and total_S to total systolic strain.

TABLE 3
Myocardial deformation parameters of the study population

| | First group* | | | | | | |
|---|---|---|---|---|---|---|---|
| | (n = 16) | | | | | | |
| | All subjects | | | | | | |
| | of the first | Dobutamine group (a subset | | Passive leg-lift group | | Anthracycline group^{†} | |
| | group | (n = 8) of the first group) | | (n = 11) | | (n = 7) | |
| | (n = 16) | BL | LD | BL | LL | BL | FU |
| PreS, % | 6.7 ± 2.49 | 6.83 ± 2.34 | 7.29 ± 2.24 | 5.96 ± 1.72 | 8.61 ± 2.18^{‡} | 8.11 ± 1.03 | 8.59 ± 1.73 |
| Total_S, % | −20.15 ± 4.49 | −20.44 ± 3.89 | −24.24 ± 5.55^{‡} | −19.65 ± 3.77 | −24.05 ± 3.67^{‡} | −21.23 ± 2.93 | −21.49 ± 2.89 |

Stretch - strain relationship

| | | | | | | | |
|---|---|---|---|---|---|---|---|
| Intercept | −10.52 ± 3.14 | −10.17 ± 2.39 | −6.5 ± 4.73^{‡} | −11.29 ± 2.34 | −11.29 ± 4.04 | −7.76 ± 3.37 | −13.97 ± 2.66^{‡} |
| Slope | −1.45 ± 0.28 | −1.47 ± 0.36 | −2.34 ± 0.36^{‡} | −1.39 ± 0.57 | −1.51 ± 0.38 | −1.68 ± 0.15 | −0.86 ± 0.23^{‡} |

*individuals used to define normal prestretch-strain relationship,
^{†}patients with breast cancer undergoing treatment with cardiotoxic anthracycline,
^{‡}p < 0.05 against the baseline of the same group.
BL—baseline,
LD—low dose dobutamine,
LL—passive leg-lift,
FU—follow-up after 3 cycles of chemotherapy,
preS—stretch of myocardial segment during the atrial contraction,
total_S—total systolic strain.

A typical example of the response of the stretch-strain relationship to the treatment with anthracycline is given in FIG. 3b. This significant decrease of the slope of preS-total_S relationship after the chemotherapy was observed in 6 out of 7 patients (FIG. 5b). The mean slope of the preS-total_S regression lines decreased significantly from −1.68±0.15 to −0.86±0.23 (p<0.05) and the mean intercept increased from −7.76±3.37 to −13.97±2.66 (p<0.05) (table 3, FIG. 4c).

In embodiments of the present invention we have related segmental systolic LV strain to segmental stretch of myocardium during atrial contraction to obtain a regional stretch-strain relationship. We have shown that the slope of this relationship gets steeper in response to a dobutamine challenge, does not change with preload induced increase of LV function and flattens after the exposure to a cardiotoxic drug. It may thus be serve as an index of LV inotropy.

### Presence of Regional Stretch-Strain Relationship in the Healthy LV

As expected from Frank-Starling law, longitudinal myocardial systolic shortening was closely related to longitudinal stretch during atrial contraction in healthy individuals. The normal stretch-strain regression equation, obtained by echocardiography through 18 segmental values in our study (total_S=−10.52−1.45*PreS) was nearly identical to the one reported by Zwanenburg for the circumferential deformation of the LV measured with tagged MRI in healthy subjects (y=1.4x+14.7), as described in Zwanenburg J J et al. Am. J. Physiol. Heart Circ. Physiol. 2005 (288) H 787-94. The low range of individual intercept and slope values, as well as a good reproducibility of regression equations even with different imaging techniques confirm that LV systolic strain dependency from stretch during atrial contraction is not a coincidental finding. It shows that in a healthy individual a major part of variability of systolic strain within the ventricle can be attributed to segmental differences in passive stretch during atrial contraction.

The presence of such relationship in the LV suggests that the Frank-Starling mechanism should not be regarded only as a global phenomenon and that it truly applies on a regional level as well. This is also apparent from numerous experimental studies that have investigated and described the underlying cellular mechanisms of the Frank-Starling phenomenon. According to these studies, passive stretching of myocardial sarcomeres increases their sensitivity to Ca2+, which results in more force generated at a given Ca2+ concentration, i.e. at a given inotropic state, as described e.g. in Holubarsch et al. in Circulation 1996 (94) 683-689, in Hibberd M G et al. in The Journal of physiology 1982 (329) pages 527-540, and in Konhilas et al. Pflugers Archiv: European journal of physiology 2002 (445) p 305-310. In-vivo regional differences of passive stretch and strain are naturally present, in spite of little variation between myocardial fiber mechanics in different LV walls, as described in Itoh A. et al. in Am. J. Physiol. Heart. Circ. Physiol. 2012 (302) H180-187. In fact, this heterogeneity of segmental passive stretch, as described by Choi H F. et al. in Journal of biomechanics 2010 (43) p1745-1753, and strain values, as described by Choi HF et al. in Am. J. Physiol. Heart. Circ. Physiol. 2011 (301) H2351-2361, at a given global LV preload seems to result from the local differences in wall curvature and thickness as demonstrated in simulation studies on the interplay between myocardial mechanics and ventricular shape. It should be noted that for the regional stretch-strain relationship, passive LV stretch during atrial contraction was used as a measure of preload therefore assuming that the myocardium is at its minimal stress state during diastasis, as described by Pasipoularides A. et al. in Circulation 1986 (74) p991-1001. As such, the relative change of LV segmental length during atrial contraction was considered as a non-invasive equivalent for the passive stretch of the myocardial fibers measured in the experimental studies on the basic mechanisms of the Frank-Starling law, as described by Konhilas et al. Pflugers Archiv: European journal of physiology 2002 (445) p 305-310.

### The Slope of Regional Stretch-Strain Relationship as an Estimate of Myocardial Inotropic State

In-vitro studies have shown that with increased inotropy the stretch-force relationship gets steeper, as described e.g. in Holubarsch et al. in Circulation 1996 (94) 683-689, in Hibberd MG et al. in The Journal of physiology 1982 (329) pages 527-540, and in Konhilas et al. Pflugers Archiv: European journal of physiology 2002 (445) p 305-310. As such the steepening of the stretch-strain relationship slope observed during the dobutamine challenge in our study was not unexpected. Moreover, these results are in an agreement with the findings of the in-vivo study performed more than 20 years ago by Glower and colleagues in Circulation 1985 (71) p994-1009. They were one of the first ones to report a close linear relationship between the end-diastolic length of myocardial segment and regional stroke work, the slope of which was getting steeper with increasing LV inotropy. Of course, neither the isometric force measured in the in-vitro experiments nor the regional stroke work measured in the in-vivo setting can be directly replaced by the systolic strain that we used in our study. We presumed that the slope of stretch-strain relationship describes the changes of myocardial inotropic state the same way as end-diastolic length-regional stroke work relationship does.

On the other hand, passive leg-lift induced preload increase did not change the slope of stretch-strain relationship. The significant increase of global LV systolic function parameters, such as LV SV, LV EF and segmental systolic strain values during the leg-lift, was thus mainly determined by increased passive myocardial stretch during the atrial contraction, and not by changes of LV inotropy. This was confirmed by the significant correlation between the change of segmental preS and the change of total_S with the passive leg lift observed in this group (cf. FIG. 5).

In contrast to the dobutamine challenge, the exposure to a cardiotoxic anthracycline resulted in a significant flattening of the slope of the stretch-strain relationship, suggesting its capability to detect the decreased LV intrinsic inotropic state. From experimental studies it is known that therapy with this drug induces myocyte death and disruption of the sarcomere structure, as described by Sawyer D B et al. in Progress in cardiovascular diseases 2010 (53) p105-113 and in Lim C C. et al. in The Journal of biological chemistry 2004 (279) p8290-8299. As such, any fall of systolic cardiac function in patients undergoing chemotherapy can likely be attributed to the impairment of LV inotropy, especially if no previously known cardiac pathology is present and if the loading conditions of the heart are normal and not changing with the treatment. This is consistent with the decrease of LV EF seen in this group of patients at follow-up, even though the latter remained within the limits of normality. This is not unexpected as early stages of anthracycline induced cardiac damage are usually subclinical and not detectable with conventional echocardiographic tools, as described in Jurcut R. et al. in Journal of the American Society of Echocardiography: official publication of the American Society of Echocardiography 2008 (21) p1283-1289. Nevertheless, even though in this small cohort of patients no change was seen in mean preS or total_S values, a significant decline of the stretch-strain relationship slope was observed in 6 out of 7 patients. Thus, this method seems to be advantageous over conventional deformation analysis when subtile changes of LV inotropy have to be detected.

It should also be pointed out, that in the patients with the breast cancer the slopes of stretch-strain relationship at the baseline were slightly steeper and LVEF slightly higher than in the other groups of healthy subjects in our study. This might indeed indicate an enhanced inotropic state caused by increased sympathetic stimulation due to the malignant process. In fact, it would also explain higher heart rates seen in those patients already at baseline. On the other hand, higher LV EF might simply be a result of smaller LV volumes in this group of female patients. In any case, this should not change the interpretation of our results, as all the individual slopes of stretch-strain relationships in the breast cancer patients at baseline were within the range of normal values seen in the healthy volunteers in our study.

These results according to embodiments of the invention provide a strong argument for the hypothesis that the steepening of the stretch-strain relationship slope is specific to the increase of LV inotropic state, whereas the flattening of it is capable to detect the decrease of LV inotropy. All of this suggests that stretch-strain relationship can be regarded as a non-invasive measure of LV inotropic state.

A non-invasive and easily applicable method, according to embodiments of the invention, for the estimation of myocardial inotropy is currently lacking in clinical practice, in particular due to the load dependency of conventional parameters of LV systolic function. The stretch-strain relationship according to embodiments of the invention advantageously can be easily extracted from adapted myocardial strain curves obtained by any deformation imaging technique, such as TDI, 2D speckle tracking or MRI tagging, which makes it an attractive tool to be used in the routine. This relationship can be obtained in any patient at rest. The interpretation of the results does not require any additional interventions, such as leg lift, Valsalva maneuver or pharmacological infusions.

The stretch-strain relationship according to embodiments of the invention can potentially serve in clinical routine, when a detection of deteriorating intrinsic LV function is important. Our results suggest that it might be used to detect cardiotoxicity in patients undergoing chemotherapy. Besides that, it could possibly be applied for the follow up of patients with mitral or aortic valve regurgitation as in those patients early detection of decreasing myocardial inotropy is crucial for the correct timing of surgical intervention, as described by Vahanian A. et al. in European heart journal 2007 (28) p230-268. Moreover, it might be a beneficial parameter to monitor the treatment of heart failure patients or to differentiate physiological forms of LV hypertrophy from the pathological ones. All of those potential implications of LV stretch-strain relationship remain the topics for future studies.

However, the gold standard to assess intropic state (i.e. analysis of ESPVR) could not be used as a reference method in this study because of its invasive nature. Therefore, an assumption had to be made that contractility was normal in all included individuals. Furthermore, in other embodiments of the present invention it was presumed that segmental myocardial inotropy was homogeneous within each ventricle, as the slopes of regional stretch-strain relationships had to give a measure of global intraventricular inotropic state. However, as only subjects without evidence of coronary heart disease were included to this study, regional inhomogeneities of LV function were unlikely. Secondly, in this embodiments of the invention we did not evaluate the effect of afterload on stretch-strain relationship because of the rapid positive inotropic response of the LV to an acute afterload increase, as described by Monroe R G et al. in The Journal of clinical investigation 1972 (51) 2573-83. It should also be mentioned, that in the group of subjects who underwent the passive leg-lift the regression lines were drawn through only three segmental stretch and strain values, as obtaining all 18 segments in those individuals was not practical due to the short duration of the preload increasing effect of this maneuver. However, we still feel confident about the regression equations obtained in that study group, as individual intercepts and slopes were very close to the ones obtained in other study subjects through 18 segmental values. Finally, this method is preferably not used in patients with atrial arrhythmias or high heart rates with fusion of mitral inflow E and A waves, since a precise separation between active LV relaxation (early diastole) and passive LV stretch (late diastole) is required. The effect of increased filling pressures, decreased LV compliance and dyssynchrony on the presence of passive LV stretch and on the applicability of stretch-strain relationship needs further detailed investigations as well.

It is to be understood that this invention is not limited to the particular features of the means and/or the process steps of the methods described as such means and methods may vary. It is also to be understood that the terminology used herein is for purposes of describing particular embodiments only, and is not intended to be limiting. It must be noted that, as used in the specification and the appended claims, the singular forms “a” “an” and “the” include singular and/or plural referents unless the context clearly dictates otherwise. It is also to be understood that plural forms include singular and/or plural referents unless the context clearly dictates otherwise. It is moreover to be understood that, in case parameter ranges are given which are delimited by numeric values, the ranges are deemed to include these limitation values.

All publications and patents mentioned herein are hereby incorporated by reference in their entirety as if each individual publication or patent was specifically and individually indicated to be incorporated by reference. In case of conflict, the present application, including any definitions herein, will control.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method for determining myocardial inotropic state, comprising the steps:
receiving a value of stretch for different myocardial segments during passive filling;
receiving associated values representative of total systolic strain; and
using a relationship between the stretch of different myocardial segments during passive filling and the associated values representative for total systolic strain as an index of the myocardial inotropic state.

2. The method according to claim 1, whereby using a relationship comprises using a slope of the functional relationship between the stretch of different myocardial segments during passive filling and the associated values representative for total systolic strain as an index of the myocardial inotropic state.

3. The method according to claim 1, wherein during said passive filling is during said atrial contraction.

4. The method according to claim 1, wherein during said passive filling is during ventricular contraction applied to the atria.

5. The method according to claim 1, whereby said total systolic strain is a total shortening of the myocardial segment, whereby said total shortening is expressed as strain difference between peak late diastolic strain and end-systolic strain values.

6. The method according to claim 1, whereby receiving a value of stretch for different myocardial segments and receiving associated values representative of total systolic strain comprises obtaining said values for said different segments in a non-invasive way.

7. The method according to claim 6, wherein obtaining said values for said different segments in a non-invasive way comprises obtaining said values for said different segments based on myocardial deformation imaging.

8. The method according to claim 1, whereby said myocardial inotropic state is the left ventricular isotropic state.

9. The method according to claim 1, whereby said method provides distinguishing between a healthy and failing contractile state of a ventricle.

10. The method according to claim 1, wherein the method comprises setting a reference point of strain curves to zero at the beginning of a P wave on an ECG, measuring a segmental prestretch of the LV as a peak positive strain during atrial contraction, and measuring systolic strain as a difference between the peak positive strain value and a peak negative systolic strain.

11. A system for determining myocardial inotropic state, the system comprising
an input means for receiving a value of stretch of different myocardial segments during passive filling and receiving associated values representative of total systolic strain; and
a processing means programmed for determining a relationship between the stretch of the different myocardial segments during passive filling and the associated values representative for total systolic strain and for using the relationship as an index of the myocardial inotropic state.

12. A system according to claim 11, wherein the input means is adapted for receiving a value of stretch of different myocardial segments during atrial contraction or during ventricular contraction applied to the atria and wherein said processing means is programmed for determining a relationship between the stretch of the different myocardial segments during atrial contraction or during ventricular contraction applied to the atria and the associated values.

13. A system according to claim 11, wherein the said processing means is programmed for determining a slope of a functional relationship between the stretch of the different myocardial segments during passive filling and the associated values.

14. A system according to claim 11, wherein the processor furthermore comprises a decision unit for distinguishing, based on the index, between a healthy and failing contractile state of a ventricle.

15. A computer program comprising computer program code means adapted to perform all the steps of a method according to claim 1 when the computer program is run on a computer.

16. The computer program according to claim 15 embodied on a computer readable medium.

17. Use of a relationship between the stretch of different myocardial segments during passive filling and associated values representative for the total systolic strain as an index of the myocardial inotropic state.

18. Use of a relationship according to claim 17, wherein the passive filling is atrial contraction.

19. Use of a relationship according to claim 17, wherein the passive filling is ventricular contraction applied to the atria.

20. Use according to claim 17, whereby said relationship is a slope of the functional relationship between the stretch of different myocardial segments during passive filling and the associated values representative for total systolic strain.
